(19) United States
(12) Patent Application Publication
(10) Pub. No.: US 2013/0296768 A1
(43) Pub. Date: Nov. 7, 2013

(54) HYDROGEN SUIT FOR SKIN EXPOSURE

(71) Applicant: Microlin, LLC., Salt Lake City, UT (US)

(72) Inventors: Ashok V. Joshi, Salt Lake City, UT (US); Jeremy Heiser, Salt Lake City, UT (US)

(21) Appl. No.: 13/888,081

(22) Filed: May 6, 2013

Related U.S. Application Data
(60) Provisional application No. 61/643,047, filed on May 4, 2012.

(51) Int. Cl. A61M 35/00

(52) U.S. Cl. CPC A61M 35/00
USPC 604/23

(57) ABSTRACT

A system facilitates creating a controlled hydrogen rich environment around a patient. The system includes an enclosure, at least one opening, and a hydrogen source. The enclosure forms an internal space to at least partially enclose a body part of the patient. The opening is defined within the enclosure to allow a portion of the body part of the patient to extend out of the internal space of the enclosure. The opening is further configured to form a seal around the body part that extends out of the internal space of the enclosure. The hydrogen source delivers hydrogen gas into the internal space of the enclosure.

### CROSS-REFERENCE TO RELATED APPLICATIONS

This application claims the benefit of U.S. Provisional Application No. 61/643,047, filed on May 4, 2012, which is incorporated by reference herein in its entirety.

### BACKGROUND

Radiation therapy is one of the primary treatment modalities for cancer patients, with about one half of all cancer patients receiving some type of radiation therapy during the course of their treatment. Radiation therapy uses high-energy radiation to kill cancer cells by damaging their DNA. The radiation used for cancer treatment can come from an implanted radioactive material located near the tumor cells, can be injected into the bloodstream, or can come from a machine outside of the body. Radiation is also used in a variety of other medical procedures.

In addition to killing cancer cells, radiation can also damage healthy, normal cells. The damage of healthy tissue is typically categorized as acute radiation side effects. These side effects are caused by damage to rapidly dividing normal cells in the area being treated and can lead to skin irritation or damage at regions exposed to the radiation beams. Radiation also may result in the replacement of normal tissue with scar tissue, leading to restricted movement of the affected area and further breakdown. The potential side effects of radiation treatment are carefully considered by the radiation oncologist and weighed against the potential benefit for each patient.

### SUMMARY

Embodiments of a system are described. In one embodiment, the system is a system to create a controlled hydrogen rich environment around a patient. An embodiment of the system includes an enclosure, at least one opening, and a hydrogen source. The enclosure forms an internal space to at least partially enclose a body part of the patient. The opening is defined within the enclosure to allow a portion of the body part of the patient to extend out of the internal space of the enclosure. The opening is further configured to form a seal around the body part that extends out of the internal space of the enclosure. The hydrogen source delivers hydrogen gas into the internal space of the enclosure. Other embodiments of the system are also described.

Embodiments of an apparatus are also described. In one embodiment, the apparatus is a hydrogen suit for a patient to wear. An embodiment of the hydrogen suit includes an external barrier layer, an internal patient interface layer, and an exhaust port. The external barrier layer is a material that is substantially impermeable to hydrogen gas. The internal patient interface layer is a material that facilitates a three-dimensional distribution of the hydrogen gas around an enclosed body part of the patient. The exhaust port facilitates controlled passage of air out of an enclosed space within the external barrier layer and the internal patient interface layer. Other embodiments of the apparatus are also described.

Embodiments of a method are also described. In one embodiment, the method is a method for creating a hydrogen rich environment for a patient. An embodiment of the method includes donning an enclosure on a body part of a patient. The method also includes sealing a gap between a perimeter of an opening in the enclosure and a portion of the patient's body extending through the opening. Sealing the gap defines a substantially closed space around the patient's body part within the enclosure. The method also includes delivering hydrogen gas into the enclosure to expose the patient's enclosed body part to a hydrogen rich environment. Other embodiments of the method are also described.

Other aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

FIG. 1A depicts one embodiment of a hydrogen suit system with a hydrogen suit connected to an external hydrogen generator.

FIG. 1B depicts another embodiment of a hydrogen suit system with a hydrogen suit which includes an integrated hydrogen generator.

FIG. 2A depicts another embodiment of a hydrogen suit system with a connection to an external hydrogen tank.

FIG. 2B depicts another embodiment of a hydrogen suit system with a connection to an external hydrogen supply source.

FIG. 3 depicts one embodiment of multi-layer suit construction.

FIG. 4A depicts one embodiment of a collar sealing member.

FIG. 4B depicts a cutaway view of another embodiment of the collar sealing member of FIG. 4A installed on a collar of a hydrogen suit.

FIG. 5 depicts another embodiment of a hydrogen suit system that includes an expansion control shell.

FIG. 6 depicts another embodiment of a hydrogen suit system that includes a series of expansion control bands.

FIG. 7 depicts another embodiment of a hydrogen suit system that includes adjustable expansion control members.

FIG. 8 depicts one embodiment of a hydrogen treatment chamber to enclose a hydrogen environment around a patient's upper body.

FIG. 9 depicts another embodiment of an adjustable hydrogen treatment chamber to enclose a hydrogen environment around a patient's torso.

FIG. 10 depicts a cutaway view of an embodiment of a hydrogen treatment chamber with an internal spacer pad.

FIG. 11 depicts one embodiment of a hydrogen hyperbaric chamber.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

While many embodiments are described herein, at least some of the described embodiments include a hydrogen (H₂) suit to reduce the acute radiation side effects associated with the breakdown of a patient's skin due to external source radiation exposure. In other words, embodiments of the hydrogen suit create and maintain a hydrogen rich environment that is in intimate contact with the user's skin for use in mitigating the acute radiation side effects associated with radiation therapy. More than a half of the ionizing radiation-induced cellular damage is caused by hydroxyl radicals, which cause skin damage and compromised skin integrity. Reducing hydroxyl radicals can significantly improve the protection of cells from radiation damage. Embodiments of the hydrogen suit will effectively facilitate exposing the patient's skin to a high concentration hydrogen environment. This environment will function to reduce the hydroxyl radicals in the patient's skin and help to prevent breakdown resulting from hydroxyl radicals.

Embodiments of the hydrogen suit present advancements in the art for several reasons. In some embodiments, the hydrogen suit allows for the application of a high concentration hydrogen environment for use during radiation therapy. In some embodiments, the environment is between about 0.000055% and 100% hydrogen. In other embodiments, the environment is between about 0.05% and 50% hydrogen. In other embodiments, the environment is between about 2% and about 4% hydrogen. It also allows for the use of a high barrier material and sealing features to create a closed environment over a portion of the patient's skin. In one embodiment, the addition of a patient interface layer allows for the free flow of hydrogen throughout the suit environment. Some embodiments of the hydrogen suit also may include the integration of restrictive elements to control the expansion of the suit and may have a feature for evacuating air from the suit prior to and/or during hydrogen delivery.

While various arrangements and constructions of the hydrogen suit are possible, some embodiments of the hydrogen suit include a barrier layer, a patient interface layer, one or more extremity sealing features, a collar sealing feature, and/or a gas delivery port.

In one embodiment, the barrier layer includes a flexible layer of material that functions to contain the hydrogen gas. Thus, the barrier layer may be substantially impermeable to hydrogen. In one embodiment, the patient interface layer includes a flexible, porous material that allows the hydrogen to come in contact with the patient's skin, even while under compression.

In one embodiment, the sealing features or elements include flexible and conformable elements to seal the system to the extremities (e.g., arms and/or legs), torso, and/or neck of the patient such that the internal environment is free of major gas leaks.

In one embodiment, the gas delivery port allows for the introduction of hydrogen into the suit environment from an external source. The external hydrogen source may include a hydrogen generator, a canister (i.e., tank), or an in-house supply system capable of supplying high concentration hydrogen. In some embodiments, a connection tube provides a path for the gas to flow from the hydrogen source to the hydrogen delivery port. In other embodiments, hydrogen may be generated internally within the hydrogen suit.

In some embodiments, the hydrogen suit also includes donning features. The donning features include one or more openings that allow the patient to enter the suit environment and extend their extremities and head out of the suit.

Embodiments of the hydrogen suit also may include environment controls to allow for automatic or manual control of the conditions of the suit environment. The environment controls may include sensors, gauges, valves, regulators, and/or other similar electrical, mechanical, or electromechanical devices.

In some embodiments, the hydrogen suit also includes suit expansion controls. The suit expansion controls limit the expansion of the suit. This reduces the volume of hydrogen needed within the suit to create the hydrogen rich environment.

Several embodiments of hydrogen suit systems are illustrated in the appended figures and described in more detail below. These configurations convey various structures, functions, and features which may be combined together as illustrated, combined together in other configurations, or implemented separately within various embodiments of the hydrogen suit systems.

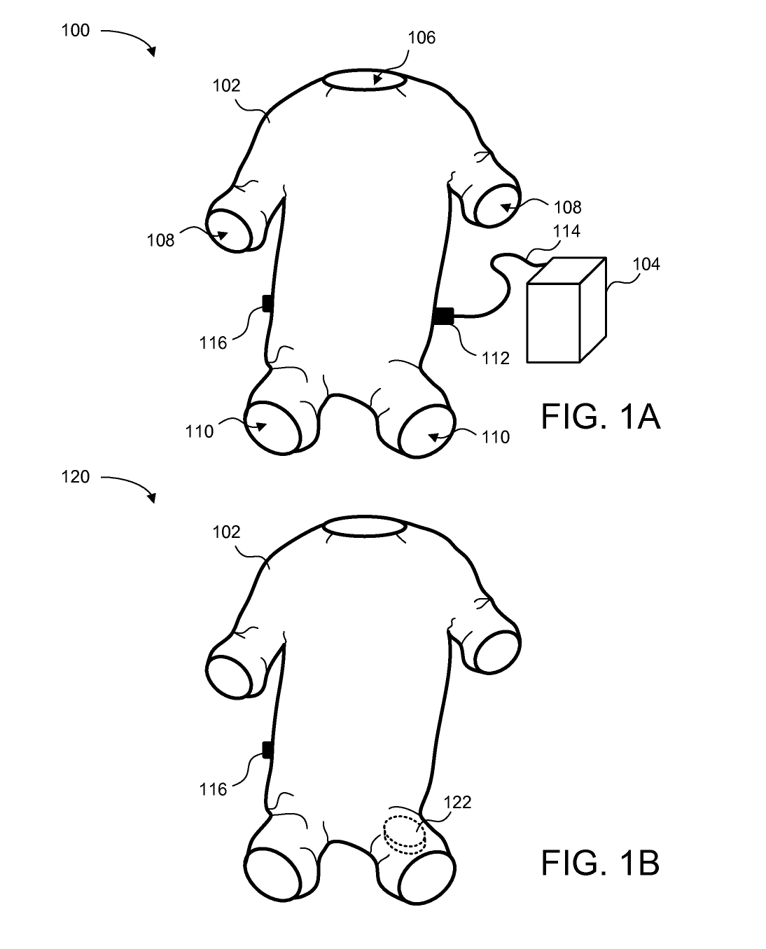

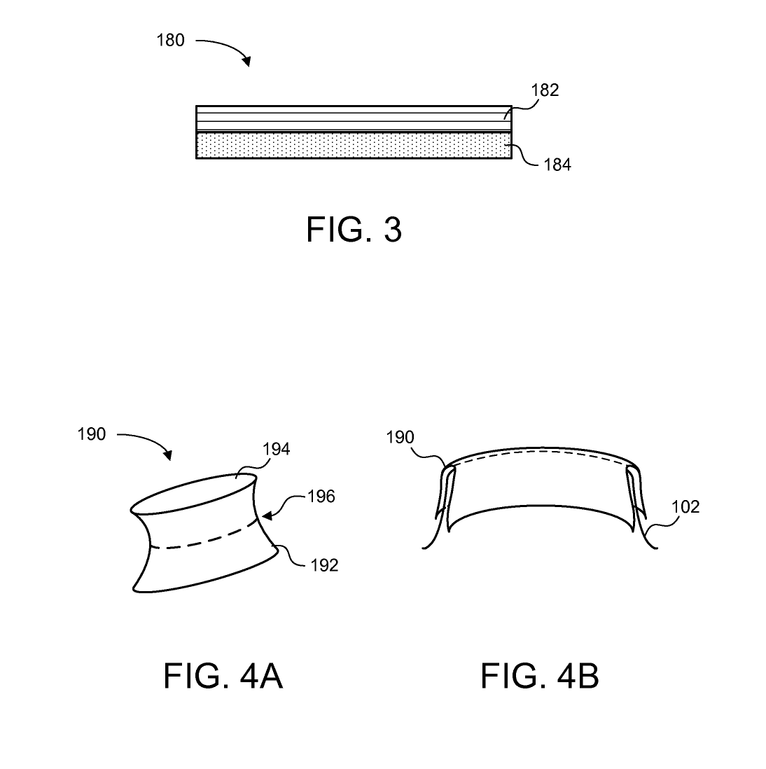

FIG. 1A depicts one embodiment of a hydrogen suit system 100 with a hydrogen suit 102 connected to an external hydrogen generator 104. In one embodiment, the hydrogen suit 102 is fabricated from a multi-layer suit construction that includes a barrier layer and a patient interface layer. One example of this type of construction is illustrated in FIG. 3 and described in more detail below. In other embodiments, the hydrogen suit 102 may be fabricated from a single layer of material or from at least one different material other than the barrier layer and the patient interface layer. For convenience, the material (e.g., one or more layers) used for the hydrogen suit may be referred to herein as the suit material.

The suit material of the depicted hydrogen suit 102 is arranged to form a plurality of openings. For example, the depicted hydrogen suit 102 includes a collar opening 106, arm openings 108, and leg openings 110. In a more specific example, the collar opening 106 of the hydrogen suit 102 opens to allow the patient to enter the suit and then closes in a manner that allows the suit to seal around the surface of the patient's neck. Other embodiments may include other quantities or types of openings (e.g., a torso opening).

Each of the openings may form an extremity sealing feature or element in order to allow the corresponding part of the patient to extend out of the hydrogen suit 102 while maintaining a close (or substantially closed) system for the internal suit environment. In some embodiments, the sealing features or elements may include silicone, neoprene, urethane, or a similar material. In some embodiments, one or more of the sealing members may include elastic features or adjustable features that would be capable of generating the force required to seal the environment to the patient's skin. Some examples of adjustable features include, but are not limited to, straps, ties, latches, locking strips, adhesive, Velcro, and so forth. Adjustable features may be used in combination with or independent of elastic features.

The illustrated hydrogen suit 102 also includes a gas delivery port 112. A connection tube 114 supplies hydrogen gas from the external hydrogen generator 104 to the internal space within the hydrogen suit 102 via the gas delivery port 112. This provides hydrogen gas to the internal suit environment for the delivery of hydrogen to any affected sites. In some embodiments, the gas delivery port 112, the connection tube 114, and/or the hydrogen generator 104 may incorporate one or more sensors, gauges, valves and/or regulators.

The illustrated hydrogen suit 102 also includes an exhaust port 116. The exhaust port 116 may function to evacuate excess air from the internal suit environment. In some embodiments, the exhaust port 116 has a power source (or a connection to a power source) to evacuate the air from the internal suit environment.

The air may be evacuated prior to introduction of the hydrogen into the hydrogen suit 102. Alternatively, the air may be exhausted in response to increased pressure resulting from the introduction of hydrogen into the hydrogen suit 102. Limiting or controlling the amount of air within the suit may allow an operator to more precisely control the quantity and/or concentration of the hydrogen that is delivered into the hydrogen suit 102. In some embodiments, the exhaust port 116 is part of an evacuation system which may include, but is not limited to, an electric pump, a manual pump, and/or a vacuum port.

FIG. 1B depicts another embodiment of a hydrogen suit system 120 with a hydrogen suit 102 which includes an integrated hydrogen generator 122. The integrated hydrogen generator 122 is internally incorporated into the hydrogen suit such that it is in gaseous communication with the internal suit environment. Similar to the external hydrogen generator shown in FIG. 1A, the integrated hydrogen generator 122 generates hydrogen gas which is circulated by or otherwise delivered to a patient's skin within the hydrogen suit 102. In some embodiments, the hydrogen may be generated using a zinc-air cell, water electrolysis, fuel pellets containing a hydrogen-generating compound such as ammonia borane, and other ways know in the art to generate hydrogen. In some embodiments, the integrated hydrogen generator 122 may utilize an internal power source. Alternatively, the integrated hydrogen generator 122 may utilize power from an external source, in which case a power port may be provided that is accessible from the outside of the hydrogen suit 102.

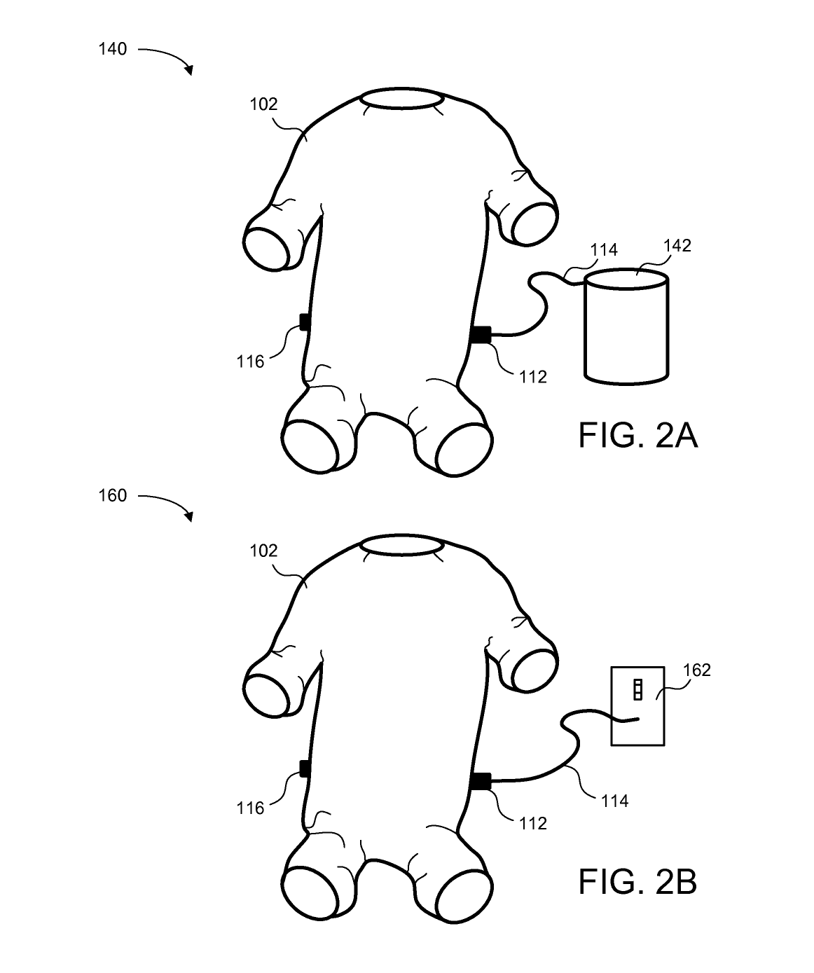

FIG. 2A depicts another embodiment of a hydrogen suit system 140 with a hydrogen suit 102 connected to an external hydrogen tank 142. The illustrated hydrogen suit system 140 is substantially similar to the hydrogen suit system 100 of FIG. 1A, except that the external hydrogen tank 142 is a source of hydrogen that does not rely on local production. Rather, the external hydrogen tank 142 is a storage canister to store hydrogen that has been made at a separate location.

FIG. 2B depicts another embodiment of a hydrogen suit system 160 with a hydrogen suit 102 connected to an external hydrogen supply source 162. In one embodiment, the external hydrogen supply source is a locally controlled connection to an in-house supply of hydrogen, for example, within a hospital or other patient treatment facility. The connection tube 114 provides a connection between the in-house supply 162 and the gas delivery port 112. The flow may be controlled by a bi-pole or variable flow switch at the source 162 and/or a flow controller at the gas delivery port 112.

Although the illustrations of FIGS. 1A, 1B, 2A, and 2B, and the descriptions, each include a single hydrogen source, other embodiments of the hydrogen suit 102 may include or be connected to a plurality of separate hydrogen sources. When a plurality of hydrogen sources are used together, the hydrogen sources may be of the same type or of different types. In further embodiments, a hydrogen source may be configured with tubing or other delivery channels to deliver hydrogen into the hydrogen suit 102 at multiple locations, in varying quantities, at varying flow rates, or under otherwise disparate operating parameters.

FIG. 3 depicts one embodiment of multi-layer suit construction 180. The illustrated multi-layer suit construction includes two functional layers: a barrier layer 182 and a patient interface layer 184. Other embodiments of the multi-layer suit construction 180 may include fewer or more layers. Additionally, other layers may be included that provide aesthetic value, but do not necessarily serve a functional purpose relative to the hydrogen gas within the hydrogen suit. The barrier layer 182 and the patient interface layer 184 are substantially permeable to radiation. In one embodiment, the patient interface layer 184 is permeable to hydrogen so that the gas contacts the skin.

Embodiments of the hydrogen suit 102 may be constructed of one or more film layers, a laminated series of films, or co-extruded film. Examples of materials include, but are not limited to: nylon, polyethylene, polypropylene, polyester, polyethylene terephthalate, polyvinyl chloride, polyvinylidene chloride, polystyrene, polyurethane, silicone, natural rubber, synthetic rubber, Barex, Topas, and so forth.

In one embodiment, the barrier layer 182 is a flexible or semi-rigid material that is capable of presents a substantial barrier to hydrogen permeation. In this way, the barrier layer 182 creates a substantially sealed or closed environment within the hydrogen suit 102. The patient interface layer 184 is a flexible or semi-rigid material that allows for distribution of the hydrogen gas within the hydrogen suit 102. In some embodiments, the patient interface layer 184 is a porous material with a thickness that presents three-dimensional distribution of the hydrogen gas around the enclosed portion of the patient's body.

FIG. 4A depicts one embodiment of a collar sealing member 190. In general, the collar sealing member 190 is a substantially impermeable material that is flexible enough to fold over the perimeter of the collar opening 106 in the hydrogen suit 102. Examples of such materials include, but are not limited to, neoprene, silicone, urethane, and so forth. In some embodiments, the collar sealing member 190 may incorporate one or more features that allow the collar opening 106 of the hydrogen suit 102 to be tightly secured to a structurally reinforced area, thereby allowing the material around the collar opening 106 to intimately interface with the material of the collar sealing member 190. Similar sealing features may be implemented to seal other extremity openings or torso openings.

The depicted collar sealing member 190 has three regions, including a base region 192, a top region 194, and a fold region 196. The base region 192 is generally configured to go inside of the collar opening 106 in the hydrogen suit 102, such that that inner surface of the base region 192 is in contact with the skin or clothing of the patient. The collar sealing member 190 may be positioned so that the fold region 196 is approximately aligned with the perimeter of the collar opening 106 in the hydrogen suit 102. So when the collar sealing member 190 is folded at approximately the fold region 196, the top region of the collar sealing member 190 folds over the perimeter of the collar opening 106 and the surrounding material of the hydrogen suit 102. FIG. 4B depicts a cutaway view of another embodiment of the collar sealing member 190 of FIG. 4A installed on a collar of a hydrogen suit 102.

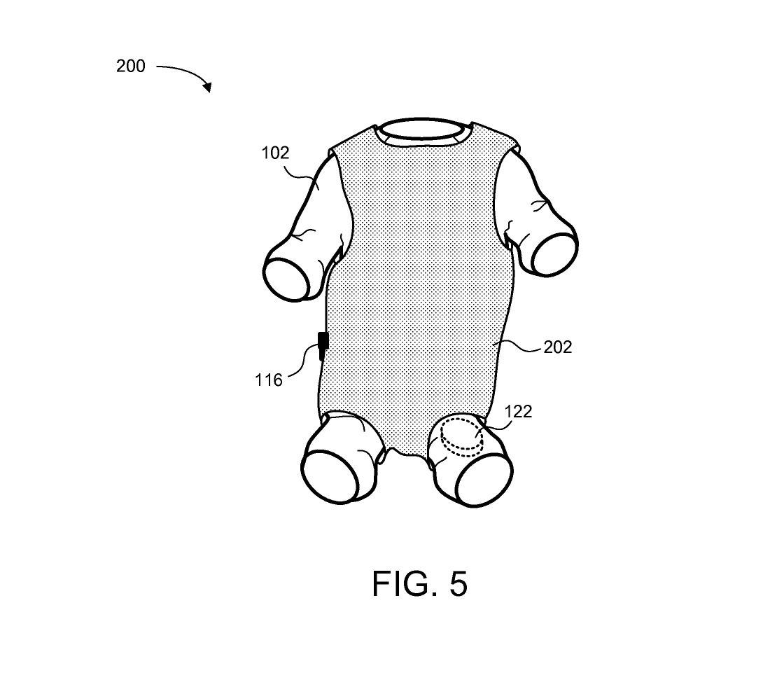

FIG. 5 depicts another embodiment of a hydrogen suit system 200 that includes a hydrogen suit 102 with an expansion control shell 202. In one embodiment, the expansion control shell 202 is a rigid or semi-rigid form that is placed over the patient once the patient is in a prone or supine position on a treatment table (not shown). The expansion control shell 202 limits the amount of inflation of the hydrogen suit 102. In this way, the volume of hydrogen used within the suit is limited, while still providing a hydrogen rich environment. Additionally, the expansion control shell 202 may be substantially permeable to radiation. Examples of such materials include, but are not limited to, polyethylene, polypropylene, polyester, polyethylene terephthalate, polyvinyl chloride, polyvinylidene chloride, polystyrene, polycarbonate, acrylic, wood, or another similar material or combination of materials.

In other embodiments, the expansion control shell 202 may circumscribe the patient's torso. Alternatively, multiple separate pieces may be combined and fastened together around the patient to form the expansion control shell 202. In some embodiments, the expansion control shell 202 may have an opening for the exhaust port 116. In another embodiment, the expansion control shell 202 may have other openings. In further embodiments, the expansion control shell 202 may cover more or less of the patient's torso, extremities, and/or head.

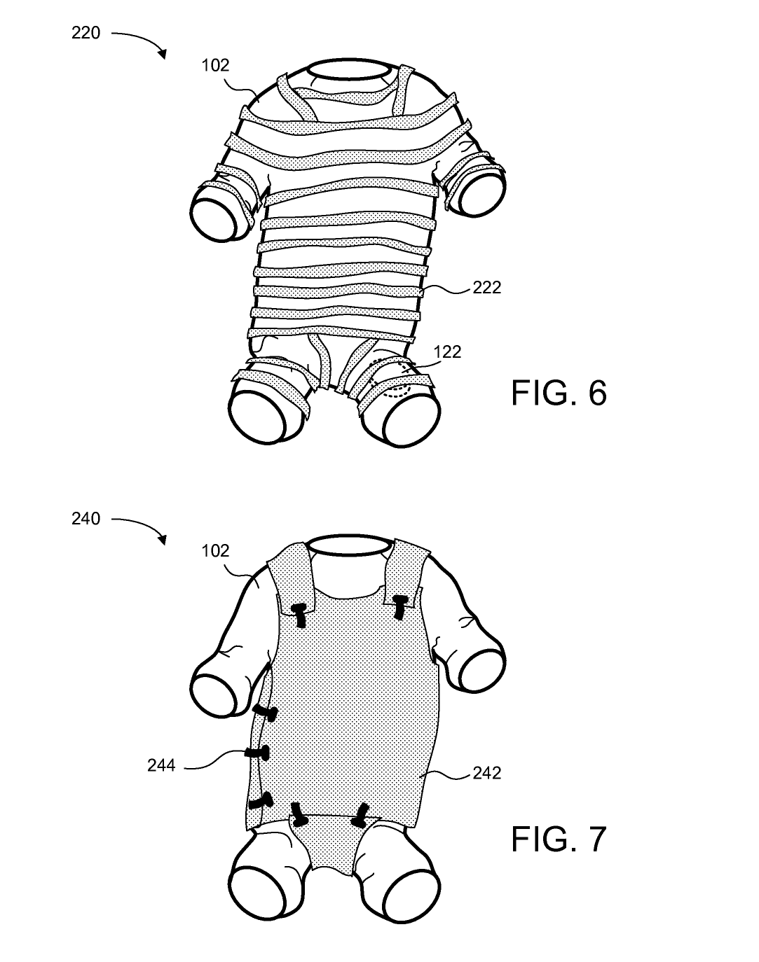

FIG. 6 depicts another embodiment of a hydrogen suit system 220 that includes a hydrogen suit 102 and a series of expansion control bands 222. The expansion control bands 222 are areas of reinforcement that function to resist excessive expansion of the hydrogen suit 102 due to the hydrogen gas. Excessive expansion may interfere with the radiological procedure, cause discomfort to the patient, or require an additional hydrogen to be delivered. In some embodiments, the expansion control bands 222 are made of nylon, polypropylene, or other similar materials. Although the expansion control bands 222 are shown on the outside of the hydrogen suit 102, in other embodiments some or all of the expansion control bands 222 may be incorporated internally within the hydrogen suit 102. Also, the specific number of expansion control bands 222 may vary depending on the size and shape of the hydrogen suit 102. Moreover, the arrangement of the expansion control bands 222 may vary depending on the size and shape of the hydrogen suit 102. In further embodiments, other types of bands, webbing, netting, or other materials with relatively high tensile strength may be used as expansion control means. In some embodiments, each band 222 or set of bands 222 may have an adjustable fastener (not shown) to individually or collectively adjust the size of the corresponding strap(s) 222.

In further embodiments, the expansion control bands 222 may be fabricated from strings, fibers, thicker areas of plastic, or other similar materials and/or structures. Additionally, the expansion control bands 222 may be included in the material lamination or secured to the material with adhesive.

FIG. 7 depicts another embodiment of a hydrogen suit system 240 that includes a hydrogen suit 102 with adjustable expansion control members 242. In the depicted embodiment, the expansion control members 242 include a torso member, shoulder straps, and a groin strap. The torso member may be adjustable with respect to the patient's torso shape and size. Similarly, the shoulder straps and the groin strap may be adjustable with respect to the patient's girth and height. Any form of fasteners 244 may be used. In some embodiments, the fasteners 244 are adjustable. In some embodiments, the fasteners 244 are substantially permeable to radiation.

In further embodiments, the expansion control members 242 may include a material that is wrapped around a patient and the hydrogen suit 102, and is further secured such that the expansion of the hydrogen suit 102 is limited. Some examples of materials include, but are not limited to a fabric or cloth, an elastomeric fabric or cloth, a mesh, netting, a plastic film, or a reinforced plastic film. The shoulder and groin straps may be made of the same or different material and may be secured to the torso member using any suitable securement method. Some examples of securement methods include, but are not limited to, straps, ties, latches, locking strips, adhesive, Velcro, and similar fasteners. In some embodiments, the expansion control members 242 may be a weighted cloth or sheet of plastic. Some examples include, but are not limited to, a blanket, a layer of canvas, and a lead x-ray vest or blanket.

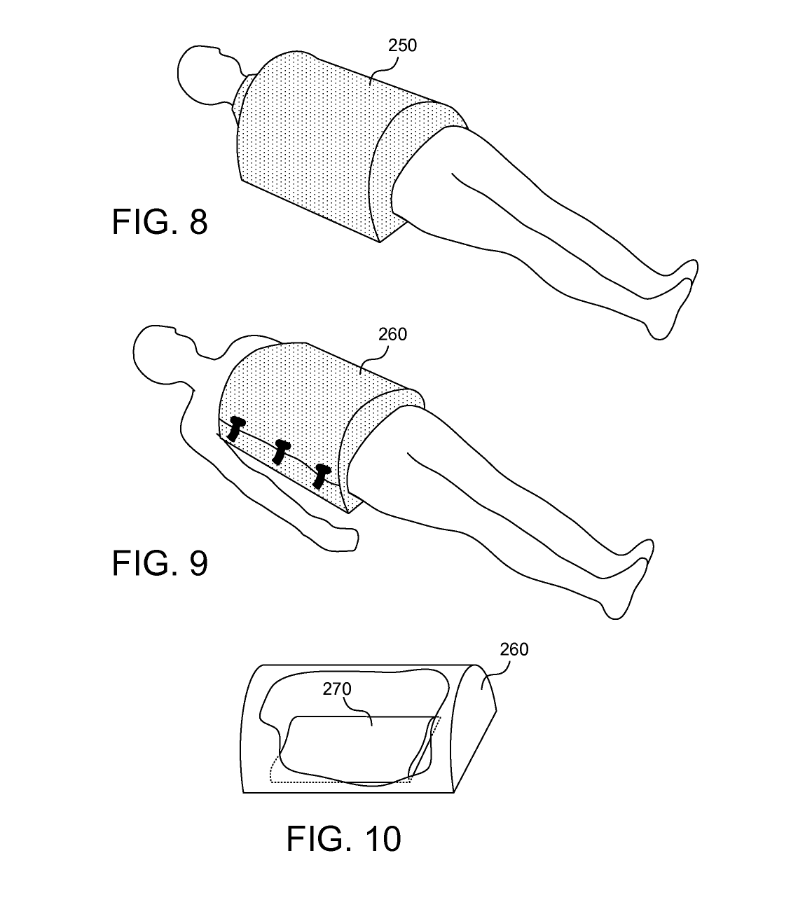

FIG. 8 depicts one embodiment of a hydrogen treatment chamber 250 to enclose a hydrogen environment around a patient's upper body. In one embodiment, the chamber 250 is made of a rigid or semi-rigid material. The chamber 250 contains the treatment area of the patient and maintains the hydrogen in the internal enclosure environment. In one embodiment, the chamber 250 is substantially sealed at the patient's lower torso to create a closed system. Similarly, the chamber is substantially sealed at the patient's neck, for example, using the collar sealing member 190. Entry into the hydrogen treatment chamber 250 may be performed by placing the chamber 250 over the patient's head and lowering the chamber 250 around the patient's torso.

In further embodiments, the hydrogen rich environment may be achieved through the use of a semi-rigid or rigid enclosure. The enclosure may closely match the contour of the patient's body or be fabricated from an alternate geometry. In some embodiments, the enclosure may have a flat or contoured bottom that is connected to an arched top or rectangular shape, or a multitude of other shapes. The bottom panel or structure may include a hinge and/or closure element to allow the patient to enter the enclosure. Some examples of materials that might be used to make the enclosure include a rigid or semi-rigid plastic such as polyethylene, polypropylene, polyester, polyethylene terephthalate, polyvinyl chloride, polyvinylidene chloride, polystyrene, polycarbonate, or acrylic. Alternatively, wood or another similar material may be used. Although not shown in detail, hydrogen may be supplied to the chamber 250 by any type of hydrogen source described herein, or by another type of hydrogen source.

FIG. 9 depicts another embodiment of an adjustable hydrogen treatment chamber 260 to enclose a hydrogen environment around a patient's torso. The depicted chamber 260 is similar, in some ways, to the chamber 250 shown in FIG. 8. However, the chamber 260 shown in FIG. 9 includes an entry flap that can be opened to allow easy entry, and then closed and adjusted with one or more fasteners. Internal frame supports may conform to the patient's shape and size. Additionally, in the depicted embodiment, the chamber 260 fits around the patient's torso, while allowing the patient's arms to move freely outside of the chamber 260. In other embodiments, the chamber 260 may be smaller or larger to accommodate different parts of the patient's body.

In further embodiments, the hydrogen rich environment may be achieved through the use of a tent-like enclosure structure that is fabricated using a support frame and a barrier material. The support frame may be constructed of semi-rigid poles, rods, or tubing. The frame may be constructed from a variety of materials and in a multitude of geometries. In some embodiments, the support frame is collapsible or made of one or more pieces. The barrier material may include, but is not limited to, nylon, polyethylene, polypropylene, polyester, polyethylene terephthalate, polyvinyl chloride, polyvinylidene chloride, polystyrene, polyurethane, silicone, natural rubber, synthetic rubber, Barex, Topas, or another similar material. Although not shown in detail, hydrogen may be supplied to the chamber 260 by any type of hydrogen source described herein, or by another type of hydrogen source.

FIG. 10 depicts a cutaway view of an embodiment of a hydrogen treatment chamber, such as the chamber 260, with an internal spacer pad 270. In one embodiment, the internal spacer pad is a flexible or semi-rigid material that allows for three-dimensional distribution of the hydrogen gas around the patient's body, including between the patient's back and the treatment table.

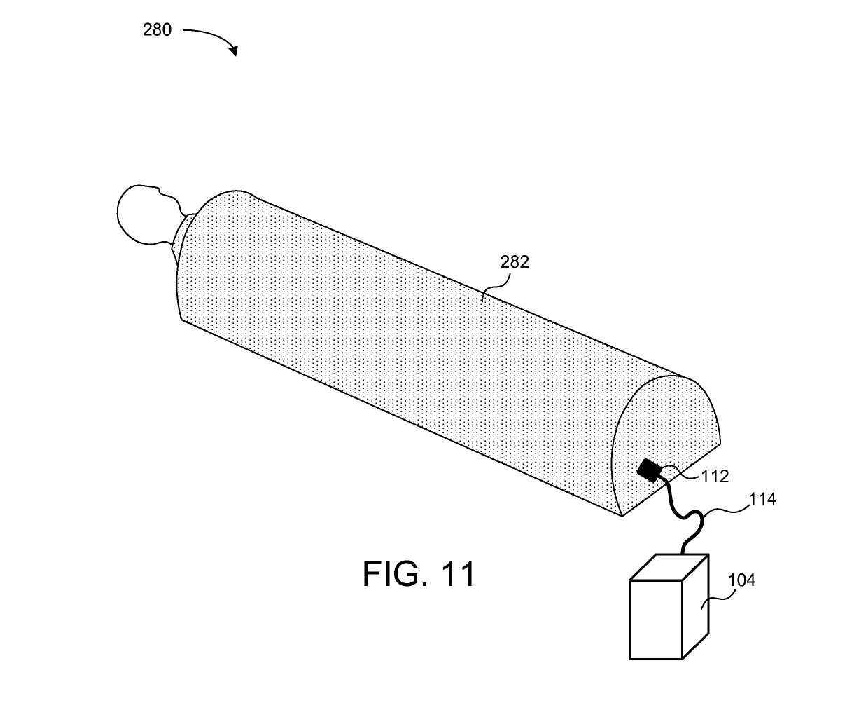

FIG. 11 depicts one embodiment of a hydrogen hyperbaric chamber system 280. The depicted hydrogen hyperbaric chamber system 280 includes a hydrogen source 104 connected to a hydrogen hyperbaric chamber 282. Although a hydrogen generator 104 is depicted, other embodiments may use different types of hydrogen sources. In general, the hydrogen hyperbaric chamber 282 provides a pressurized internal chamber that exposes the patient to higher pressure hydrogen atmosphere. Similar to some of the other embodiments described herein, the depicted embodiment utilizes a sealing member around the patient's neck or other body part below the patient's head. This allows the body to be exposed to the higher pressure hydrogen atmosphere, while allowing the head to be exposed to standard pressure atmosphere. Furthermore, the hydrogen hyperbaric chamber 280 may be fabricated of a material that is substantially permeable to radiation.

Although many embodiments depicted and described herein relate to covering a patient's torso, other embodiments may be implemented to cover other portions of the patient's body. For example, in one embodiment, the hydrogen suit covers some or all of a patient's hand, arm, foot, or leg.

In some embodiments, various types of monitoring and/or control devices may be incorporated into the hydrogen suit 102 and/or the surrounding components. Some embodiments may include temperature control/feedback device(s) for controlling the temperature of the hydrogen that the patient is exposed to. Some embodiments may include flow rate control/feedback device(s) for controlling the flow rate of the hydrogen into the hydrogen suit 102 or otherwise incident on the skin of the patient. These and other similar types of devices may be implemented for the benefit of the patient's personal comfort.

Although the embodiments described above focus on the use of hydrogen gas, other embodiments may use a mixture of hydrogen and another gas, or another gas that excludes hydrogen, etc.

Embodiments of the hydrogen suit 102 have potential uses including, but not limited to, protecting a patient's skin from the side effects associated with radiation therapy, protecting a patient's skin from the side effects associated with procedures that involve long duration radiation exposure, or protecting a patient's skin from the side effects associated with repeated radiation exposure.

In some embodiments, a topical product such as a cream may be applied to the skin beneath the hydrogen suit or on body surfaces not covered by the suit. The topical product may be formulated such that it neutralizes, either wholly or partially, free radicals. One such cream is described in United States Patent Publication No. 20090074735, which is incorporated herein by reference.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A system comprising:
an enclosure to form an internal space configured to at least partially enclose a body part of a patient;
at least one opening defined within the enclosure, wherein the opening is configured to allow a portion of the body part of the patient to extend out of the internal space of the enclosure, wherein the opening is further configured to form a seal around the body part that extends out of the internal space of the enclosure; and
a hydrogen source coupled to the enclosure to deliver hydrogen gas into the internal space of the enclosure.

2. The system of claim 1, wherein the enclosure is conformable to the enclosed body part of the patient.

3. The system of claim 1, wherein the at least one opening comprises a collar opening configured to form a seal around the patient's neck.

4. The system of claim 3, further comprising a collar sealing member configured to form a substantial barrier to passage of the hydrogen gas out of the enclosure at the collar opening.

5. The system of claim 4, wherein the collar sealing member comprises:
a base region configured to fit through the collar opening and inside of the enclosure;
a fold region; and
a top region configured to fold, at the fold region, back over a perimeter of the collar opening on an outside portion of the enclosure, wherein pressure from the base region and/or the top region creates the substantial barrier to passage of the hydrogen gas between the collar sealing member and the patient's neck.

6. The system of claim 1, wherein the at least one opening comprises an extremity opening configured to form a seal around the patient's hand, arm, foot, or leg.

7. The system of claim 1, further comprising a gas delivery port coupled to the enclosure, wherein the gas delivery port is configured to allow the hydrogen gas to pass through a controlled opening into the enclosure.

8. The system of claim 1, further comprising a hydrogen source to deliver the hydrogen gas into the internal space of the enclosure.

9. The system of claim 8, wherein the hydrogen source comprises an external hydrogen source.

10. The system of claim 9, wherein the external hydrogen source comprises an external hydrogen generator.

11. The system of claim 9, wherein the external hydrogen source comprises an external hydrogen storage tank.

12. The system of claim 9, wherein the external hydrogen source comprises a house connection to hydrogen within a building system.

13. The system of claim 9, further comprising a connection tube to connect the external hydrogen source to the gas delivery port.

14. The system of claim 8, wherein the hydrogen source comprises an integrated hydrogen source that is located within the enclosure.

15. The system of claim 14, wherein the integrated hydrogen source comprises an internal hydrogen generator.

16. The system of claim 1, further comprising an exhaust port coupled to the enclosure, wherein the exhaust port is configured to allow air to exhaust from within the enclosure to the ambient environment.

17. The system of claim 16, further comprising a power source to actively evacuate air from within the enclosure during or prior to delivery of the hydrogen gas into the enclosure.

18. The system of claim 1, wherein the enclosure comprises a multi-layer construction with a barrier layer and a patient interface layer, wherein:
the barrier layer comprises a material that is substantially impermeable to the hydrogen gas; and
the patient interface layer comprises a material that facilitates a three-dimensional distribution of the hydrogen gas around the patient's enclosed body part.

19. The system of claim 18, wherein the barrier layer and the patient interface layer are substantially permeable to radiation.

20. The system of claim 1, wherein the enclosure is substantially permeable to radiation.

21. The system of claim 1, further comprising an expansion control member to constrain expansion of the enclosure despite increased pressure from the hydrogen gas within the enclosure.

22. The system of claim 21, wherein the expansion control member comprises an expansion control shell, wherein the expansion control shell comprises a rigid or semi-rigid form configured to at least partially surround the enclosure.

23. The system of claim 21, wherein the expansion control member comprises a plurality of expansion control bands, wherein the expansion control bands are attached to the enclosure and configured to at least partially surround the enclosure.

24. The system of claim 21, wherein the expansion control member comprises a plurality of adjustable expansion control members that, when coupled together, are configured to at least partially surround the enclosure.

25. The system of claim 1, wherein the enclosure comprises a rigid or semi-rigid chamber.

26. The system of claim 25, wherein the chamber comprises a tent structure that is adjustable around the patient's torso.

27. The system of claim 25, wherein the chamber comprises a hyperbaric hydrogen chamber to enclose a patient's body, except for the patient's head.

28. The system of claim 1, wherein the enclosure is further configured to confine the hydrogen gas within the internal space to maintain a hydrogen rich environment around the enclosed body part of the patient.

29. The system of claim 1, wherein the hydrogen rich environment comprises from about 0.000055% to about 100% hydrogen.

30. The system of claim 29, wherein the hydrogen rich environment comprises from about 0.05% to about 50% hydrogen.

31. The system of claim 30, wherein the hydrogen rich environment comprises from about 2% to about 4% hydrogen.

32. A hydrogen suit for wearing on a patient, the hydrogen suit comprising:
an external barrier layer comprising a material that is substantially impermeable to hydrogen gas;
an internal patient interface layer comprising a material that facilitates a three-dimensional distribution of the hydrogen gas around an enclosed body part of the patient; and
an exhaust port to facilitate controlled passage of air out of an enclosed space within the external barrier layer and the internal patient interface layer.

33. The hydrogen suit of claim 32, further comprising a gas delivery port to facilitate controlled passage of the hydrogen gas into an enclosed space within the external barrier layer and the internal patient interface layer.

34. The hydrogen suit of claim 32, further comprising a sealing member configured to form a substantial barrier to passage of the hydrogen gas out of an opening in the external barrier layer and the internal patient layer through which a portion of the patient's body extends.

35. The hydrogen suit of claim 32, wherein the external barrier layer and the internal patient interface layer are substantially permeable to radiation.

36. The hydrogen suit of claim 32, further comprising expansion control members to constrain expansion of the hydrogen suit despite increased pressure from the hydrogen gas within the hydrogen suit.

37. The hydrogen suit of claim 32, further comprising an integrated hydrogen generator within the enclosed space of the external barrier layer, wherein the integrated hydrogen generator is configured to generate hydrogen within the enclosed space of the external barrier layer.

38. A method comprising:
donning an enclosure on a body part of a patient;
sealing a gap between a perimeter of an opening in the enclosure and a portion of the patient's body extending through the opening, wherein sealing the gap defines a substantially closed space around the patient's body part within the enclosure; and
delivering hydrogen gas into the enclosure to expose the patient's enclosed body part to a hydrogen rich environment.

39. The method of claim 38, further comprising exposing the patient's enclosed body part to a hydrogen rich environment simultaneously with exposure of the patient to radiation therapy.
